(19) United States
(12) Patent Application Publication
(10) Pub. No.: US 2024/0148779 A1
(43) Pub. Date: May 9, 2024

(54) METHODS FOR THE TREATMENT OF PROTEINOPATHIES

(71) Applicant: RENATUS INC., Yuseong-gu (KR)

(72) Inventor: Hee Gon KIM, Daejeon (KR)

(73) Assignee: RENATUS INC., Yuseong-gu (KR)

(21) Appl. No.: 18/484,111

(22) Filed: Oct. 10, 2023

Related U.S. Application Data
(60) Provisional application No. 63/415,451, filed on Oct. 12, 2022.

(51) Int. Cl. A61K 31/724; A61P 25/28; A61P 29/00

(52) U.S. Cl. CPC A61K 31/724; A61P 25/28; A61P 29/00

(57) ABSTRACT

Provided is a method of i) preventing or reducing protein aggregation, ii) reducing protein deposition, iii) reducing cytotoxicity induced by protein aggregation or deposition or deposition, or iv) alleviating or reducing inflammation induced by protein aggregation or deposition in an individual, wherein the method comprises administering a therapeutically effective amount of a pharmaceutical composition including a gamma-cyclodextrin oligomer to the individual, thereby preventing or reducing protein aggregation, reducing protein deposition, reducing cytotoxicity induced by protein aggregation or deposition, or alleviating or reducing inflammation induced by protein aggregation or deposition in the individual diagnosed with, suspected to have, or at risk of developing proteinopathy.

### BACKGROUND

Proteinopathy is a disease, disorder, and/or condition associated with protein misfolding, protein aggregation, abnormal metabolism and/or degradation of proteins. In general, proteinopatheis are linked to and/or characterized by accumulation of certain proteins and their aggregates. Protein aggregates are observed in diseases, disorders, and/or conditions, including neurodegenerative diseases, cognitive impairment disorders, ocular diseases, cardiovascular diseases, inflammatory diseases, immunologic diseases, mitochondrial diseases, and lysosomal storage diseases. Examples of proteins that get misfolded and trigger proteinopathies are beta amyloid, alpha synuclein, prion proteins, superoxide dismutase, Huntingtin and serum amyloid A.

### SUMMARY

In one aspect, the present invention provides a method of i) preventing or reducing protein (e.g., amyloid beta, alpha-synuclein, or tau) aggregation, ii) reducing protein deposition, iii) reducing cytotoxicity induced by protein aggregation or deposition or deposition, or iv) alleviating or reducing inflammation (e.g., as measured by RNA levels, intracellular or extracellular cytokine proteins, inflammation-targeted imaging modalities) induced by protein aggregation or deposition in an individual, wherein the method comprises administering a therapeutically effective amount of a pharmaceutical composition comprising a gamma-cyclodextrin oligomer to the individual, thereby preventing or reducing protein(e.g., amyloid beta, alpha-synuclein, or tau) aggregation, reducing protein deposition, reducing cytotoxicity induced by protein aggregation or deposition, or alleviating or reducing inflammation induced by protein aggregation or deposition in the individual diagnosed with, suspected to have, or at risk of developing proteinopathy.

In some embodiments, protein aggregation is reduced (e.g., as measured by RNA levels, intracellular or extracellular proteins, invasive or noninvasive molecular imaging modalities) by at least 10% or greater relative to prior to treatment with a pharmaceutical composition. In some embodiments, protein deposition is reduced by at least 10% or greater relative to prior to the treatment with a pharmaceutical composition. In some embodiments, cytotoxicity induced by protein aggregation or deposition is reduced by at least 10% or greater relative to prior to the treatment with a pharmaceutical composition. In some embodiments, inflammation (e.g., as measured by RNA levels, intracellular or extracellular cytokine proteins, inflammation-targeted imaging modalities) induced by protein aggregation or deposition is reduced by at least 10% or greater relative to prior to the treatment with a pharmaceutical composition.

In some aspects, an effective amount of a gamma-cyclodextrin oligomer may be administered to an individual. In some aspects, the gamma-cyclodextrin oligomer may comprise a mixture of two or more gamma-cyclodextrin oligomer species, wherein each of the two or more gamma-cyclodextrin oligomer species is comprised of at least 2 and at most 20 gamma-cyclodextrin monomers. In some aspects, the average molecular weight of the gamma-cyclodextrin oligomer species is from about 2.5 kDa to about 50 kDa. In some aspects, the gamma-cyclodextrin monomer is gamma-cyclodextrin or its derivatives. In some aspects, the derivative is hydroxypropyl-gamma-cyclodextrin. In some aspects, the hydroxypropyl-gamma-cyclodextrin has a molar substitution value between 0.2 and 0.9. In some aspects, the gamma-cyclodextrin oligomer composition is chemically modified by replacing a part of the structure (e.g., hydrogen) with another atom or a functional group. In some aspects, the gamma-cyclodextrin oligomer composition comprises 10% (w/w) or less of gamma-cyclodextrin monomers.

The present invention also provides a method of treating proteinopathy in an individual diagnosed with, suspected to have, or at risk of developing proteinopathy, wherein the method comprises administering a therapeutically effective amount of a pharmaceutical composition comprising a gamma-cyclodextrin oligomer to the individual. In some cases, the method comprises preventing or reducing protein (e.g., amyloid beta, alpha-synuclein, or tau) aggregation (e.g., as measured by RNA levels, intracellular or extracellular proteins, invasive or noninvasive molecular imaging modalities) by at least 10% or greater in the individual. In some cases, the treating comprises reducing protein deposition by at least 10% or greater. In some cases, the treating comprises reducing cytotoxicity induced by protein aggregation or deposition by at least 10% or greater. In some cases, the treating comprises alleviating or reducing inflammation (e.g., as measured by RNA levels, intracellular or extracellular cytokine proteins, inflammation-targeted imaging modalities) induced by protein aggregation or deposition by at least 10% or greater. In some cases, the treating alleviates, reduces, or reverses loss of neural functions.

In some aspects, the proteinopathy is a taupathy. In some embodiments, the taupathy is selected from the group consisting of Parkinson's disease, Alzheimer's disease, Lewy Body Dementia, Pick's disease, progressive supranuclear palsy, dementia pugilistica, parkinsonism linked to chromosome 17, Lytico-Bodig disease, tangle predominant dementia, Argyrophilic grain disease, ganglioglioma, gangliocytoma, meningioangiomatosis, subacute sclerosing panencephalitis, lead encephalopathy, tuberous sclerosis, Hallervorden-Spatz disease, lipofuscinosis, corticobasal degeneration, frontotemporal dementia, frontotemporal lobar degeneration, Huntington's disease, and age-related macular degeneration.

In some aspects, the proteinopathy is a synucleinopathy. In some aspects, the synucleinopathy is selected from the group consisting of Parkinson's disease, Lewy Body Dementia, multiple system atrophy, and age-related macular degeneration.

In some aspects, the proteinopathy is amyloidopathy. In some aspects, the amyloidopathy is selected from the group consisting of Alzheimer's disease, and age-related macular degeneration.

In some cases, the method prevents, reduces, or reverses the progression of dementia in the individual.

In some cases, the method prevents, reduces, or reverses loss of neural functions in the individual. In some cases, the loss of neural function involves loss of cognitive function, autonomic function, motor function, and/or eye vision.

In any one of the preceding aspects, the therapeutically effective amount is an amount sufficient to achieve the concentration of a gamma-cyclodextrin oligomer composition in a target tissue or organ from about 0.01 mg/ml to about 20 mg/ml.

The present invention also provides a method of alleviating, reducing, or reversing loss of neural functions in an individual diagnosed with, suspected to have, or at risk of developing proteinopathy, wherein the method comprises administering a therapeutically effective amount of a pharmaceutical composition comprising a gamma-cyclodextrin oligomer to the individual.

In some aspects, the loss of neural function involves loss of cognitive function, autonomic function, motor function, and/or eye vision.

In some aspects, the loss of neural function involves loss of cognitive function. In some embodiments, the method prevents, reduces, or reverses deterioration in complex attention, executive functions, memory, language, perceptual-motor control, conceptual thinking, calculations, orientation, decision making, social cognition, and/or problem solving.

In some aspects, the loss of neural function involves loss of autonomic function and the method prevents, reduces or reverses orthostatic hypotension, sialorrhea, dysphagia, nausea, hyperhidrosis and/or urinary and sexual dysfunction.

In some aspects, the loss of neural function involves loss of motor function and the method prevents, reduces or reverses Parkinsonism. In some aspects, the method prevents, reduces or reverses rest tremor, bradykinesia, rigidity, postural instability and/or impaired balance.

In some aspects, the loss of neural function involves loss of eye vision and the method prevents, reduces, or reverses reduction in central vision, need for brighter lighting, difficulty adapting to low lights, blurriness, and/or trouble recognizing faces.

In any one of the preceding aspects, the therapeutically effective amount is an amount sufficient to achieve the concentration of a gamma-cyclodextrin oligomer composition in a target tissue or organ from about 0.01 mg/ml to about 20 mg/ml.

In another aspect, a pharmaceutical composition is provided comprising a sufficient amount of a gamma-cyclodextrin oligomer composition for preventing or reducing protein aggregation, improving metabolism and/or clearance of protein aggregation, reducing cytotoxicity induced by protein aggregation or deposition, or alleviating or reducing inflammation induced by protein aggregation or deposition in the individual diagnosed with or suspected to have proteinopathy, and a pharmaceutically acceptable excipient.

In another aspect, a pharmaceutical composition is provided comprising a sufficient amount of a gamma-cyclodextrin oligomer composition to treat proteinopathy-associated diseases including, but not limited to, Alzheimer's disease, Parkinson's disease, amyloidosis, or age-related macular degeneration, and a pharmaceutically acceptable excipient.

In another aspect, a pharmaceutical composition is provided comprising a sufficient amount of a gamma-cyclodextrin oligomer composition to reduce loss of neural functions including, but not limited to, cognitive function, autonomic function, motor function, and/or eye vision, and a pharmaceutically acceptable excipient.

### BRIEF DESCRIPTION OF THE DRAWINGS

FIG. 1 shows a representative gel permeation chromatography result of cyclodextrin oligomer compositions prepared using hydroxypropyl-gamma-cyclodextrin (HPGCD) with an average molecular weight of 3.8 kDa (A) and 8.8 kDa (B).

FIG. 2 shows the matrix-assisted laser desorption ionization time of flight mass spectrometer data confirming the formation of oligomers of hydroxypropyl-gamma-cyclodextrin with an average molecular weight of 3.8 kDa (A) and 8.8 kDa (B).

FIG. 3 shows the effects of cyclodextrins, hydroxypropyl-gamma-cycloextrin (HPGCD) and HPGCD oligomer (Oligo-HPGCD) on the prevention of aggregation of amyloid beta (Aβ42), alpha-synuclein (αSyn), and Tau (huTau441) as measured by the fluorescence intensity of Thioflavin T over 24 hours. (n=3).

FIG. 4 shows the effects of cyclodextrins on the prevention of aggregation of amyloid beta (Aβ42), alpha-synuclein (αSyn), and Tau (huTau441) as measured by the fluorescence intensity of Thioflavin T at 24 hours post-incubation. Data are mean±SD. *P<0.05, **P<0.01, ***P<0.001 compared to Control; One-way ANOVA analysis and Tukey's multiple comparisons test (n=3).

FIG. 5 shows the effects of cyclodextrins on the reversal of aggregation of amyloid beta (Aβ42), alpha-synuclein (αSyn), and Tau (huTau441) as measured by the fluorescence intensity of Thioflavin T at 24 hours post-incubation. Data are mean±SD. *P<0.05, **P<0.01, ***P<0.001 compared to Control; One-way ANOVA analysis and Tukey's multiple comparisons test (n=4).

FIG. 6 shows the effects of average molecular weights of cyclodextrins on the prevention of aggregation of amyloid beta (Aβ42) as measured by the fluorescence intensity of Thioflavin T at 24 hours post-incubation. Data are mean±SD. *P<0.05, *<0.01, ***P<0.001 compared to Control; *P<0.05, **P<0.01 compared to 85.2 kDa; One-way ANOVA analysis and Tukey's multiple comparisons test (n=4).

FIG. 7 shows the effects of cyclodextrins on the cytotoxicity induced by amyloid-beta aggregation or deposition in SH-SY5Y and ARPE-19 cells. Data are mean±SD. *P<0.05, **P<0.01, ***P<0.001 compared to No treatment; One-way ANOVA analysis and Tukey's multiple comparisons test (n=4).

FIG. 8 shows the effects of cyclodextrins on the cytotoxicity induced by alpha-synuclein aggregation or deposition in SH-SY5Y cells. Data are mean±SD. *P<0.05, ***P<0.001 compared to No treatment; One-way ANOVA analysis and Tukey's multiple zo comparisons test (n=4).

FIG. 9 shows the effects of molecular weights of cyclodextrins on the cytotoxicity induced by protein aggregation or deposition in SH-SY5Y cells. Data are mean±SD. *P<0.05, **P<0.01, ***P<0.001 compared to No treatment; One-way ANOVA analysis and Tukey's multiple comparisons test (n=4).

FIG. 10 shows effects of cyclodextrins on the cytotoxicity induced by cyclodextrins in SH-SY5Y cells. Data are mean±SD. *P<0.05, **P<0.01, ***P<0.001 compared to Control; One-way ANOVA analysis and Tukey's multiple comparisons test (n=4).

FIG. 11 shows effects of cyclodextrins on the plasma membrane cholesterol extraction in SH-SY5Y cells. Data are mean±SD. **P<0.01, ***P<0.001 compared to Control; One-way ANOVA analysis and Tukey's multiple comparisons test (n=4).

FIG. 12 shows the effects of cyclodextrins on protein aggregate-induced inflammation as measured by enzyme-linked immunosorbent assay (ELISA). Data are mean±SD. ***P<0.001 compared to No treatment; One-way ANOVA analysis and Tukey's multiple comparisons test (n=4).

FIG. 13 shows the zebrafish Alzheimer's model induced by injection of Aβ42 (A), treatment protocol (B), and the therapeutic effect of oligo-HPGCD on the avoidance response (C). Data are mean±SD. *P<0.05, **P<0.01 compared to No treatment, ##P<0.01 compared to Control; Two-tailed T-test (n=20).

FIG. 14 shows the reduction of Aβ42 induced by treatment with RN-005 and donepezil (A), and representative fluorescence images (B). Data are mean±SD. *P<0.05; One-way ANOVA analysis and Tukey's multiple comparisons test (n=10)

### DETAILED DESCRIPTION OF THE DISCLOSURE

Proteinopathy is a disease, disorder, and/or condition associated with protein misfolding, protein aggregation, abnormal metabolism and/or degradation of proteins. In general, proteinopatheis are linked to and/or characterized by accumulation of certain proteins and their aggregates.

Proteionpathies are widespread through the population and cause various kinds of diseases. In Alzheimer's disease, for example, patients exhibit extracellular amyloid beta plaques and intracellular tau-containing neurofibrillary tangles in the brain. The presence of protein (e.g., amyloid beta, alpha-synuclein, or tau) aggregation, its deposition, and abnormal metabolism and/or degradation of the protein can exacerbate neuroinflammation and neuronal cell loss, contributed to the progression of the disease. Taupathies are caused of various diseases including Alzheimer's disease, progressive supranuclear palsy, Pick's disease, corticobasal degeneration, and post-encephalitic parkinsonism. Alpha-synucleins can form insoluble fibrils and accumulate in pathologic hallmark inclusions, such as Lewy body, Lewy neuritis, and glial cytoplasmic inclusions. Alpha-synucleins can induce mitochondrial dysfunction, inflammation, and neuronal cell death, causing various diseases including Parkinson's disease, Lewy body dementia, and multiple system atrophy. Age-related macular degeneration (AMD) is the most common eye disease in elderly patients, which can lead to progressive and irreversible vison loss. Accumulating data suggests that the accumulation of amyloid beta in the retina causes inflammation and macular degeneration. Cataract is caused by protein misfolding and aggregation in the eye lens, which makes cloudy area in the lens and leads to a decrease in vision.

Cyclodextrins are cyclic oligosaccharides that are comprised of glucopyranose subunits linked by α-1,4 glycosidic bonds. Depending on the number of glucoses comprising a cyclodextrin molecule, cyclodextrin is classified into alpha-, beta-, and gamma-cyclodextrin, which has six, seven, and eight glucopyranose subunits, respectively. They exhibit ring-like structure with relatively hydrophobic cavity and relatively hydrophilic surface. Therefore, binding of cyclodextrins to molecules (e.g., chemicals, peptides, proteins) harboring hydrophobic moieties can increase the solubility of the molecules, thereby preventing their aggregation and/or improving their transport, metabolism, or clearance. Cyclodextrin also has many derivatives wherein the hydroxyl groups are substituted with other functional groups. One of its derivatives, hydroxypropyl-beta-cyclodextrin, is in clinical trials for the treatment of varying cholesterol-driven zo diseases including Niemann-Pick Type C, and Alzheimer's disease. However, hydroxypropyl-beta-cyclodextrin can cause disruption of plasma membrane via cholesterol extraction thereof, which can lead to its dose-limiting side effect, ototoxicity, in an individual.

Disclosed herein are methods for preventing or reducing protein aggregation, reducing cytotoxicity induced by protein (e.g., amyloid beta, alpha-synuclein, or tau) aggregation, reducing protein deposition (e.g., as measured by RNA levels, intracellular or extracellular cytokine proteins, inflammation-targeted imaging modalities), and/or alleviating or reducing inflammation induced by protein aggregation or deposition in an individual. In some cases, the methods involve treating proteinopathy (e.g., taupathy, synucleinopathy, or amyloidopathy by e.g., slowing down, halting, or reversing the progression of the proteinopathy) in an individual diagnosed with, suspected to have, or at risk of developing proteinopathy. In some cases, the methods involve alleviating, reducing, or reversing loss of neural functions in an individual diagnosed with, suspected to have, or at risk of developing proteinopathy.

Generally, the methods provided herein involve administering a therapeutically effective amount of a pharmaceutical composition comprising a gamma-cyclodextrin oligomer to the individual in need thereof. In some embodiments, the gamma-cyclodextrin oligomer composition may comprise a mixture of two or more gamma-cyclodextrin oligomer species, wherein each of the two or more gamma-cyclodextrin oligomer species is comprised of at least 2 and at most 20 gamma-cyclodextrin monomers. In some embodiments, the average molecular weight of the gamma-cyclodextrin oligomer species is from about 2.5 kDa to about 50 kDa. In some embodiments, the gamma-cyclodextrin monomer is gamma-cyclodextrin or its derivatives. In some embodiments, the derivative is hydroxypropyl-gamma-cyclodextrin. In some embodiments, the gamma-cyclodextrin oligomer composition is chemically modified by replacing a part of the structure with another atom or a functional group. In some embodiments, the gamma-cyclodextrin oligomer composition comprises 10% (w/w) or less of gamma-cyclodextrin monomers.

In one aspect, a gamma-cyclodextrin oligomer composition prevents or reverses protein (e.g., amyloid-beta, alpha-synuclein, Tau) aggregation, improve transport, zo metabolism, and/or degradation of the protein, which will result in reduction of protein aggregation or deposition, protein aggregate-induced cytotoxicity, inflammation, and/or neural cell death, and thus progression of proteinopathy and its consequences. Consequentially, delaying, preventing, or treating proteinopathy through prevention of proteinopathy will result in alleviating, reducing, or reversing loss of neural functions in an individual diagnosed with, suspected to have, or at risk of developing proteinopathy.

### Definitions

The articles “a” and “an” are used in this disclosure to refer to one or more than one (e.g., at least one) of the grammatical object of the article, unless the context clearly dictates otherwise. By way of example, “an element” means one element or more than one element.

The term “and/or” is used in this disclosure to mean either “and” or “or” unless indicated otherwise

The terms “subject,” “individual,” and “patient” are used interchangeably and include any animal, including mammals, e.g., humans, mice rats, rabbits, dogs, cats, swine, cattle, sheep, horse, or other primates.

The term “about” a number refers to that number plus or minus 10 of that number. The term “about” a range refers to that range plus 10% of its greatest value and minus 10% of its lowest value.

The terms “treat,” “treating,” or “treatment,” or other grammatical equivalents include any effect, for example, alleviating, reducing, modulating, ameliorating, or eliminating, that results in the improvement of the condition, disease, disorder, and the like, or alleviating one or more symptoms thereof. Treating can be curing, improving, or at least partially ameliorating the disorder. In certain embodiments, treating is curing the disease.

The term “pharmaceutically acceptable” includes molecular entities and formulations that are generally safe, non-toxic, and neither biologically nor otherwise undesirable and is acceptable for pharmaceutical use in humans and non-human animals.

The term “pharmaceutically acceptable excipient” and “pharmaceutically acceptable zo carrier” refer to a substance that helps the administration of an active ingredient to and absorption by a subject and can be included in the compositions of the present invention without causing a significant adverse effect in the patient. Non-limiting examples of pharmaceutically acceptable excipients include water, NaCl, saline solutions, alcohols, oils, gelatins, carbohydrates such as lactose, amylose or starch, fatty acid esters, hydroxyethyl cellulose, polyvinyl pyrrolidine, and colors, and the like.

The terms “effective amount” or “therapeutically effective amount” refer to the amount of a compound or composition sufficient to effect beneficial or desired results. An effective amount can be administered in one or more administration, applications, or dosages and is not intended to be limited to a particular formulation or administration route.

The gamma-cyclodextrin defined in the present specification is a cyclic oligosaccharide in which eight glucopyranose units are bonded by α-(1,4)-glycosidic bonds and may include gamma-cyclodextrin and a derivative thereof.

The term “derivative” refers to a compound obtained by substituting a part of a structure of cyclodextrin, particularly, a hydroxyl group of C2, C3, or C6, with another atom or atomic group. A derivative may be induced by replacing at least one hydrogen in an unsubstituted mother group with another atom or a functional group.

The term “substituted” refers to that a part of a structure of cyclodextrin (e.g., hydrogen) is substituted with at least one substituent selected from halides, C1-C40 alkyl groups, C2-C40 alkenyl groups, C2-C40 alkynyl groups, C3-C40 cycloalkyl groups, C3-C40 cycloalkenyl groups, and C7-C40 aryl groups. When it is stated as a functional group is “selectively substituted”, it means that the functional group may be substituted with the above substituent.

### Methods of Treating Proteinopathy

Disclosed herein are the methods for treating proteinopathy in an individual having, suspected to have, or at risk of developing proteinopathy, proteinopathy-associated diseases, or loss of neural functions by preventing or reversing protein aggregation, zo and/or reducing protein (e.g., amyloid beta, alpha-synuclein, or tau) deposition (e.g., as measured by invasive or non-invasive molecular imaging modalities, immunohistochemistry), and/or improving transport, metabolism, or degradation of the protein, and/or reducing protein aggregate-induced cytotoxicity and or inflammation.

In one embodiment, treating proteinopathy in an individual as described herein prevents or reduces protein aggregation, reduces protein deposition, reduces cytotoxicity induced by protein aggregation or deposition, and/or alleviates or reduces inflammation induced by protein aggregation or deposition. The presence of protein aggregation is observed in many proteinopathy-induced diseases including Alzheimer's disease, Parkinson's disease, and age-related macular degeneration. Protein aggregation can take place through chemical or physical degradation and is dependent on the thermodynamic stability of the protein's original state. The driving force behind the formation of protein aggregation is the reduction in free surface energy through the elimination of hydrophobic residues from contact with the solvent. Since the aggregated proteins can be cytotoxic by disrupting protein degradation process, disrupting the integrity of plasma membrane, or sequestering other proteins. The protein aggregates can induce inflammation as well, exacerbating proteinopathy. Therefore, treating proteinopathy in an individual can alleviate such consequences by preventing or reversing protein aggregation.

In some cases, treating proteinopathy in an individual can reduce protein (e.g., amyloid beta, alpha-synuclein, or tau) aggregates at least about 10% or greater (e.g., at least about 15%, at least about 20%, at least about 30%, at least about 40%, at least about 50%, or greater) relative to protein aggregates prior to the treatment with a pharmaceutical composition.

In some cases, treating proteinopathy in an individual as described herein reduces protein (e.g., amyloid beta, alpha-synuclein, or tau) deposition (e.g., as measured by invasive or non-invasive molecular imaging modalities, immunohistochemistry) by at least 10% or greater (e.g., at least 15%, at least about 20%, at least about 30%, at least about 50%, or greater) relative to the protein deposition prior to the treatment with a zo pharmaceutical composition.

In some cases, treating proteinopathy in an individual as described herein reduces protein proteinopathy-associated cell death by at least 10% or greater (e.g., at least 15%, at least about 20%, at least about 30%, at least about 50%, or greater) relative to the cell death prior to the treatment with a pharmaceutical composition.

In some cases, treating proteinopathy in an individual as described herein alleviates or reduces inflammation (e.g., as measured by cytokine protein, RNA levels, inflammation-targeted imaging modalities) by at least 10% or greater (e.g., at least about 15%, at least about 20%, at least about 30%, at least about 40%, at least about 50%, or greater) relative to inflammation prior to the treatment with a pharmaceutical composition.

In one embodiment, treating in an individual as described herein treats proteinopathy.

In some instances, the proteinopathy is a taupathy. In some instances, the taupathy is selected from the group consisting of Parkinson's disease, Alzheimer's disease, Lewy Body Dementia, Pick's disease, progressive supranuclear palsy, dementia pugilistica, parkinsonism linked to chromosome 17, Lytico-Bodig disease, tangle predominant dementia, Argyrophilic grain disease, ganglioglioma, gangliocytoma, meningioangiomatosis, subacute sclerosing panencephalitis, lead encephalopathy, tuberous sclerosis, Hallervorden-Spatz disease, lipofuscinosis, corticobasal degeneration, frontotemporal dementia, frontotemporal lobar degeneration, Huntington's disease, and age-related macular degeneration.

In some instances, the proteinopathy is a synucleinopathy. In some instances, the synucleinopathy is selected from the group consisting of Parkinson's disease, Lewy Body Dementia, multiple system atrophy, and age-related macular degeneration.

In some instances, the proteinopathy is amyloidopathy. In some instances, the amyloidopathy is selected from the group consisting of Alzheimer's disease, and age-related macular degeneration.

In one embodiment, treating neural functions in an individual as described herein alleviates, reduces, or reverses loss of neural functions in the individual.

In some aspects, the loss of neural functions involve loss of cognitive function, autonomic function, motor function, and/or eye vision.

In some aspects, treating neural functions in an individual alleviates, reduces, or reverses loss of cognitive function (e.g., as evaluated by cognitive function tests), autonomic function (e.g., as evaluated by autonomic function tests), motor function (e.g., as evaluated by motor function tests), and/or eye vision (e.g., as evaluated by eye vision tests).

In some aspects, the loss of cognitive function includes, but not limited to, deterioration in complex attention, executive functions, memory, language, perceptual-motor control, conceptual thinking, calculations, orientation, decision making, social cognition, and/or problem solving. In some aspects, treating neural functions in an individual described herein reduces loss of cognitive functions by at least about (e.g., at least about 15%, at least about 20%, at least about 30%, at least about 40%, at least about 50%, or greater). In some aspects, treating neural functions in an individual described herein improves cognitive function at least about 10% or greater (e.g., at least about 15%, at least about 20%, at least about 30%, at least about 40%, at least about 50%, or greater).

In some aspects, the loss of autonomic function includes, but not limited to, orthostatic hypotension, sialorrhea, dysphagia, nausea, hyperhydrosis and/or urinary and sexual dysfunction. In some aspects, treating neural functions in an individual described herein reduces loss of autonomic functions by at least about (e.g., at least about 15%, at least about 20%, at least about 30%, at least about 40%, at least about 50%, or greater). In some aspects, treating neural functions in an individual described herein improves autonomic function at least about 10% or greater (e.g., at least about 15%, at least about 20%, at least about 30%, at least about 40%, at least about 50%, or greater).

In some aspects, the loss of motor function includes, but not limited to, rest tremor, bradykinesia, rigidity, postural instability and/or impaired balance. In some instances, the loss of eye vision includes, but not limited to, loss in central vision, need for brighter zo lighting, difficulty adapting to low lights, blurriness, and/or trouble recognizing faces. In some aspects, treating neural functions in an individual described herein reduces loss of motor functions by at least about 10% (e.g., at least about 15%, at least about 20%, at least about 30%, at least about 40%, at least about 50%, or greater). In some aspects, treating the subject described herein improves motor function at least about 10% or greater (e.g., at least about 15%, at least about 20%, at least about 30%, at least about 40%, at least about 50%, or greater).

In some instances, treating neural functions in an individual described herein reduces loss of eye vision by at least about 10% (e.g., at least about 15%, at least about 20%, at least about 30%, at least about 40%, at least about 50%, or greater). In some aspects, treating neural functions in an individual described herein improves eye vision at least about 10% or greater (e.g., at least about 15%, at least about 20%, at least about 30%, at least about 40%, at least about 50%, or greater).

In various aspects, the methods involve administering a gamma-cyclodextrin oligomer composition to an individual. Cyclodextrins are cyclic oligosaccharides that are comprised of glucopyranose subunits linked by α-1,4 glycosidic bonds. Depending on the number of glucoses comprising a cyclodextrin molecule, cyclodextrin is classified into alpha-, beta-, and gamma-cyclodextrin, which has six, seven, and eight glucopyranose subunits, respectively. They exhibit ring-like structure with relatively hydrophobic cavity and relatively hydrophilic surface. Therefore, binding of cyclodextrins to molecules (e.g., chemicals, peptides, proteins) harboring hydrophobic moieties can increase the solubility of the molecules, thereby preventing their aggregation and/or improving their transport, metabolism, or clearance. Cyclodextrin also has many derivatives wherein the hydroxyl groups are substituted with other functional groups. One of its derivatives, hydroxypropyl-beta-cyclodextrin (HPBCD), is in clinical trials for the treatment of varying cholesterol-driven diseases including Niemann-Pick Type C, and Alzheimer's disease. Most commercial HPBCDs have an average of between 4 and 9 hydroxypropyl groups. HPBCD can be selected from VTS-270/adrabetadex, Trappsol® Cyclo™, Kleptose® HP Parenteral Grade, Kleptose® HPB Parenteral Grade, Kleptose® HPB-LB Parenteral Grade, Cavitron® W7 HP5 Pharma cyclodextrin, Cavitron® W7 HP7 Pharma cyclodextrin. However, hydroxypropyl-beta-cyclodextrin can cause disruption of plasma membrane via cholesterol extraction thereof, which can lead to its dose-limiting side effect, ototoxicity, in an individual.

In various aspects, the methods provided herein involve administering a gamma-cyclodextrin oligomer composition to an individual (e.g., human, non-human animals), in need thereof (e.g., having proteinopathy, proteinopathy-induced diseases). In some cases, the individual is suspected of having or at risk of developing proteinopathy.

In some cases, a gamma-cyclodextrin oligomer composition may comprise a mixture of two or more gamma-cyclodextrin oligomer species. In some cases, each of the two or more gamma-cyclodextrin oligomer species is comprised of at least 2 and at most 20 gamma-cyclodextrin monomers. In some cases, the gamma-cyclodextrin oligomer species are formed using bifunctional crosslinking agents (e.g., bifunctional alkylating agent). In a particular embodiment, the bifunctional crosslinking agent is epichlorohydrin.

In some cases, the average molecular weight of the gamma-cyclodextrin oligomer species is from about 2.5 kDa to about 50 kDa. In some cases, the average molecular weight of the gamma-cyclodextrin oligomer species is between 2.5 kDa and 50 kDa. In some cases, the average molecular weight may be in a range of, for example, about 2.5 kDa to about 50 kDa, about 3 kDa to about 50 kDa, about 3.5 kDa to about 50 kDa, about 4 kDa to about 50 kDa, about 4.5 kDa to about 50 kDa, about 5 kDa to about 50 kDa, about 2.5 kDa to about 40 kDa, about 3 kDa to about 40 kDa, about 3.5 kDa to about 40 kDa, about 4 kDa to about 40 kDa, about 4.5 kDa to about 40 kDa, about 5 kDa to about 40 kDa, about 2.5 kDa to about 30 kDa, about 3 kDa to about 30 kDa, about 3.5 kDa to about 30 kDa, about 4 kDa to about 30 kDa, about 4.5 kDa to about 30 kDa, about 5 kDa to about 30 kDa, about 2.5 kDa to about 20 kDa, about 3 kDa to about 20 kDa, about 3.5 kDa to about 20 kDa, about 4 kDa to about 20 KDa, about 4.5 kDa to about 20 kDa, about 5 kDa to about 20 kDa, about 2.5 kDa to about 10 kDa, about 3 kDa to about 10 kDa, about 3.5 kDa to about 10 kDa, about 4 Kda to about 10 kDa, about 4.5 kDa to about 10 kDa, about 5 kDa to about 10 kDa.

In some aspects, the gamma-cyclodextrin monomer is gamma-cyclodextrin or its derivatives. In some aspects, the derivative may be induced by replacing at least one hydrogen in an unsubstituted mother group with another atom or a functional group. The functional group may be substituted with at least one substituent selected from the group consisting of halides, C1-C40 alkyl groups, C2-C40 alkenyl groups, C2-C40 alkynyl groups, C3-C40 cycloalkyl groups, C3-C40 cycloalkenyl groups, and C7-C40 aryl groups. For example, the hydrogen can be replaced with C1-C10 linear or branched alkyl, hydroxy C1-C10 linear or branched alkyl, sulfobutylether C1-C10 linear or branched alkyl, or carboxy C1-C10 linear or branched alkyl; particularly, C1-05 linear or branched alkyl, hydroxy C1-05 linear or branched alkyl, sulfobutylether C1-05 linear or branched alkyl, or carboxy C1-05 linear or branched alkyl; or more particularly, methyl, hydroxypropyl, sulfobutylether, or carboxy methyl, but embodiments are not limited thereto.

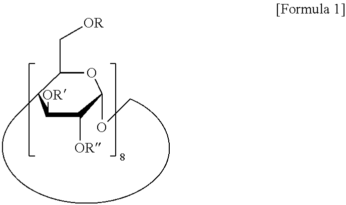

In some aspects, the gamma-cyclodextrin monomer is represented by Formula 1. R, R′ and R″ bonded to hydroxyl groups of C2, C3, and C6 of Formula 1 may be, for example, hydrogen, C1-C10 linear or branched alkyl, hydroxy C1-C10 linear or branched alkyl, sulfobutylether C1-C10 linear or branched alkyl, or carboxy C1-C10 linear or branched alkyl; particularly, may be hydrogen, C1-05 linear or branched alkyl, hydroxy C1-05 linear or branched alkyl, sulfobutylether C1-05 linear or branched alkyl, or carboxy C1-05 linear or branched alkyl; or more particularly, may be hydrogen, methyl, hydroxypropyl, sulfobutylether, or carboxy methyl, but embodiments are not limited thereto.

When the substituted hydrogen per one glucose is shown as a molar substitution, the molar substitution of the gamma-cyclodextrin derivative may be between 0.2 and 0.9. In some embodiments, the molar substitution value may be between, for example, about 0.2 to about 0.9, about 0.3 to about 0.9, about 0.4 to about 0.9, about 0.5 to about 0.9, about 0.6 to about 0.9, about 0.7 to about 0.9, about 0.2 to about 0.8, about 0.3 to about 0.8, about 0.4 to about 0.8, about 0.5 to about 0.8, about 0.6 to about 0.8, about 0.2 to about 0.7, about 0.3 to about 0.7, about 0.4 to about 0.7, about 0.5 to about 0.7, about 0.2 to about 0.6, about 0.3 to about 0.6, about 0.4 to about 0.6. In some embodiments, the derivative is hydroxypropyl-gamma-cyclodextrin with a molar substitution value zo between 0.2 and 0.9

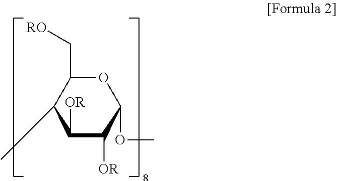

In some aspects, the hydroxypropyl-gamma-cyclodextrin comprises glucose units of the structure represented by Formula 2 wherein each R, independently for each occurrence, is hydrogen or hydroxypropyl, wherein hydroxypropyl comprises one or more hydroxypropyl groups.

In some aspects, the gamma-cyclodextrin monomer is hydroxypropyl-gamma-cyclodextrin represented by Formula 2 having a molar substitution in a range of about 0.5 to about 0.75.

In some aspects, the gamma-cyclodextrin oligomer composition is chemically modified by replacing a part of the structure (e.g., hydrogen) with another atom or a functional group selected from the group consisting of halides, C1-C40 alkyl groups, C2-C40 alkenyl groups, C2-C40 alkynyl groups, C3-C40 cycloalkyl groups, C3-C40 cycloalkenyl groups, and C7-C40 aryl groups. For example, the hydrogen can be replaced with C1-C10 linear or branched alkyl, hydroxy C1-C10 linear or branched alkyl, sulfobutylether C1-C10 linear or branched alkyl, or carboxy C1-C10 linear or branched alkyl; particularly, C1-C5 linear or branched alkyl, hydroxy C1-C5 linear or branched alkyl, sulfobutylether C1-C5 linear or branched alkyl, or carboxy C1-C5 linear or branched alkyl; or more particularly, methyl, hydroxypropyl, sulfobutylether, or carboxy methyl, but embodiments are not limited thereto.

In some cases, the gamma-cyclodextrin oligomer composition comprises 10% (w/w) or less of gamma-cyclodextrin monomers. In some cases, the gamma-cyclodextrin oligomer composition comprises less than about 10% (w/w), less than about 9% (w/w), less than about 8% (w/w), less than about 7% (w/w), less than about 6% (w/w), less than about 5% (w/w), less than about 4% (w/w), less than about 3% (w/w), less than about 2% (w/w), less than about 1% (w/w), less than about 0.5% (w/w), less than about 0.4% (w/w), less than about 0.3% (w/w), less than about 0.2% (w/w), less than about 0.1% (w/w), less than about 0.05% (w/w), or less than about 0.01% of gamma-cyclodextrin monomers.

In some cases, a gamma-cyclodextrin oligomer composition reduces protein aggregation, cytotoxicity induced by protein aggregation or deposition, and/or inflammation induced by protein aggregation more effectively than cyclodextrin monomers or their derivatives (e.g., hydroxypropyl-alpha, -beta-, or -gamma-cyclodextrin monomers).

In some cases, oligomerization of cyclodextrin results in increased affinity to proteins (e.g., amyloid beta, alpha-synuclein, or tau) due to physicochemical properties obtained by the oligomerization process (e.g., increased binding with the hydrophobic moiety of the proteins, increased solubility, increased stability, oligomer structure, etc) compared to cyclodextrin monomers, which in turn leads to more effective prevention or reduction of the protein aggregation, thereby reducing cytotoxicity and inflammation induced by the protein aggregation. In some aspects, when the average molecular weight of the gamma-cyclodextrin oligomer species is out of the range between 2.5 kDa and 50 kDa, its effects on reducing protein aggregation, cytotoxicity induced by protein aggregation or deposition, and/or inflammation induced by protein aggregation may be reduced.

In some cases, oligomerization of cyclodextrin results in less cytotoxicity (e.g., higher cell viability) induced by cyclodextrins due to physicochemical properties obtained by the oligomerization process (e.g., decrease in plasma membrane cholesterol extraction, decrease in plasma membrane disruption) compared to cyclodextrin monomers, thereby reducing cyclodextrin-mediated toxicity or adverse events.

In some aspects, a gamma-cyclodextrin oligomer composition reduces protein aggregation or deposition, improves metabolism, degradation, and/or clearance of the protein, and/or reduces cytotoxicity induced by protein aggregation or deposition in cells, tissue, or organs. In some aspects, the organs include, but not limited to, the brain, eye, liver, blood vessel, heart, spleen, lung, or skin.

In some aspects, a therapeutically effective amount of a gamma-cyclodextrin oligomer composition is an amount enough to achieve the therapeutic effect described herein.

In some aspects, the therapeutically effective amount is an amount sufficient to achieve the concentration of a gamma-cyclodextrin oligomer composition in a target tissue or organ from about 0.01 mg/ml to about 20 mg/ml.

In some aspects, the methods involve treating an individual with a combination of a gamma-cyclodextrin oligomer composition and an additional therapeutic (e.g., active pharmaceutical ingredients, medical procedure, or surgery). In some cases, the gamma-cyclodextrin oligomer composition and the additional therapeutic are administered to the subject at or near the same time (e.g., in a single formulation, or as separate formulations). In some cases, the gamma-cyclodextrin oligomer composition and the additional therapeutic are administered at different times (e.g., in separate formulations). In some cases, the additional therapeutic is administered prior to administration of the gamma-cyclodextrin oligomer composition. In some cases, the additional therapeutic is administered after administration of the gamma-cyclodextrin oligomer composition.

In some aspects, the individual may have previously been undergoing treatment with an additional therapeutic prior to administration of the gamma-cyclodextrin oligomer composition. In some cases, the treatment with the additional therapeutic may be zo ineffective or may have limited efficacy. In such cases, individuals treated with the gamma-cyclodextrin oligomer composition may exhibit a greater therapeutic benefit than administration of the additional treatment alone.

In some aspects, individuals treated with both a gamma-cyclodextrin oligomer composition and an additional therapeutic may exhibit a therapeutic benefit greater than the therapeutic benefit exhibited by treatment with either the additional therapeutic or the gamma-cyclodextrin oligomer composition alone. In some cases, treatment with both the additional therapeutic and the gamma-cyclodextrin oligomer composition has a synergistic effect, such that the interaction between the additional therapeutic and the gamma-cyclodextrin oligomer composition causes the total effect of the therapeutics to be greater than the sum of the individual effects of each therapeutic. In some aspects, treatment with both the additional therapeutic and the gamma-cyclodextrin oligomer composition has an additive effect.

### Pharmaceutical Composition

In one aspect, the present invention provides pharmaceutical compositions comprising a gamma-cyclodextrin oligomer for the treatment of proteinopathy, proteionpathy-induced diseases, and/or loss of neural functions induced by proteinopathies in an individual. In some embodiments, the individual is a human patient.

The pharmaceutical composition comprising a gamma-cyclodextrin oligomer may further include a pharmaceutical additive selected from the group consisting of a diluent, a binder, a disintegrant, a lubricant, an enhancer, and any combination thereof other than the gamma-cyclodextrin polymer. Also, the pharmaceutical composition comprising a gamma-cyclodextrin oligomer may be formulated into injections such as aqueous solutions, suspensions, emulsions, etc., pills, capsules, granules or tablets, with the aid of a diluent, a dispersant, a surfactant, a binder, or a lubricant. Also, the pharmaceutical composition comprising a gamma-cyclodextrin oligomer may further include a pharmaceutically acceptable carrier, i.e., saline, sterile water, Ringer's solution, buffered saline, cyclodextrin, a dextrose solution, a maltodextrin solution, glycerol, ethanol, liposome, or a mixture of one or more thereof, and if necessary, other common additive such as an antioxidant, a buffer, etc. Moreover, the pharmaceutical composition comprising a gamma-cyclodextrin oligomer may be formulated according to respective components using an appropriate method known in the art or a method disclosed in Remington's Pharmaceutical Science (Mack Publishing Company, Easton PA).

The diluent, which may be used to increase quantity, may be selected from the group consisting of the group consisting of mannitol, lactose, starch, microcrystalline cellulose, Ludipress®, calcium dihydrogen phosphate, and any combinations thereof, but embodiments are not limited thereto.

The binder may be selected from the group consisting of povidone, hydroxypropyl cellulose, hydroxypropyl methyl cellulose, polyvinyl alcohol, sodium carboxymethyl cellulose, and any combinations thereof, but embodiments are not limited thereto.

The disintegrant may be selected from the group consisting of croscarmellose sodium, crospovidone, sodium starch glycolate, and any combinations thereof, but embodiments are not limited thereto.

The lubricant may be selected from the group consisting of stearic acid, metal salts of stearic acid (for example, calcium stearate, or magnesium stearate), talc, colloid silica, sucrose fatty acid ester, hydrogenated vegetable oil, wax, glyceryl fatty acid esters, glycerol dibehenate, and any combinations thereof, but embodiments are not limited thereto.

The enhancer may be hyaluronidase (e.g., hyaluronidase 1-4, PH20, or HYALP1).

In some cases, the pharmaceutical composition comprising a gamma-cyclodextrin oligomer comprises at least about 1 g, at least about 2 g, at least about 3 g, at least about 4 g, at least about 5 g, at least about 6 g, at least about 7 g, at least about 8 g, at least about 9 g, at least about 9 g, at least about 10 g, at least about 25 g, at least about 50 g, at least about 75 g, at least about 100 g, at least about 150 g, or at least about 200 g of a gamma-cyclodextrin oligomer composition.

In some cases, the pharmaceutical composition comprises a 0.1% (w/v), 0.5% (w/v), 0.1% (w/v), 0.5% (w/v), 1% (w/v), 2% (w/v), 3% (w/v), 4% (w/v), 5% (w/v), 10% (w/v), 15% (w/v), 20% (w/v), 25% (w/v), 30% (w/v), 35% (w/v), 40% (w/v), 45% (w/v), or 50% (w/v) aqueous solution of a gamma-cyclodextrin oligomer.

In some cases, the pharmaceutical compositions of the present invention further comprise one or more pharmaceutically acceptable excipients. In some embodiments, the one or more pharmaceutically acceptable excipients can be selected from the group comprising a diluent, a buffering agent, a stabilizer, a solubilizing agent, a preservative, an enhancer, or any combination thereof.

In some cases, the pharmaceutical composition may be formulated for administration as a liquid dosage form suitable form suitable for intravenous, intravascular, subcutaneous, intramuscular, intrathecal, intraocular, depot, and peristaltic pump administration.

In some cases, the pharmaceutical composition may be administered by a device (e.g., wearable injection systems).

### EXAMPLES

### Example 1. Preparation of Gamma-cyclodextrin Oligomer

To induce oligomerization, gamma-cyclodextrin monomers were dissolved in NaOH solution (33% w/w) and then an appropriate amount of epichlorohydrin was added. After 24 hours, acetone was added to stop the oligomerization reaction and then acetone was removed by decantation. The remaining solution was incubated in the oven at 50° C. for 24 hours. The pH of the solution was adjusted to between 6 and 11 using hydrochloric acid. Salts, cyclodextrin monomers, and small byproducts were removed using dialysis or ultrafiltration. An average molecular weight was measured using gel permeation chromatography. Herein, hydroxypropyl-gamma-cyclodextrin (HPGCD) with a molar substitution value between 0.4 and 0.8 was used.

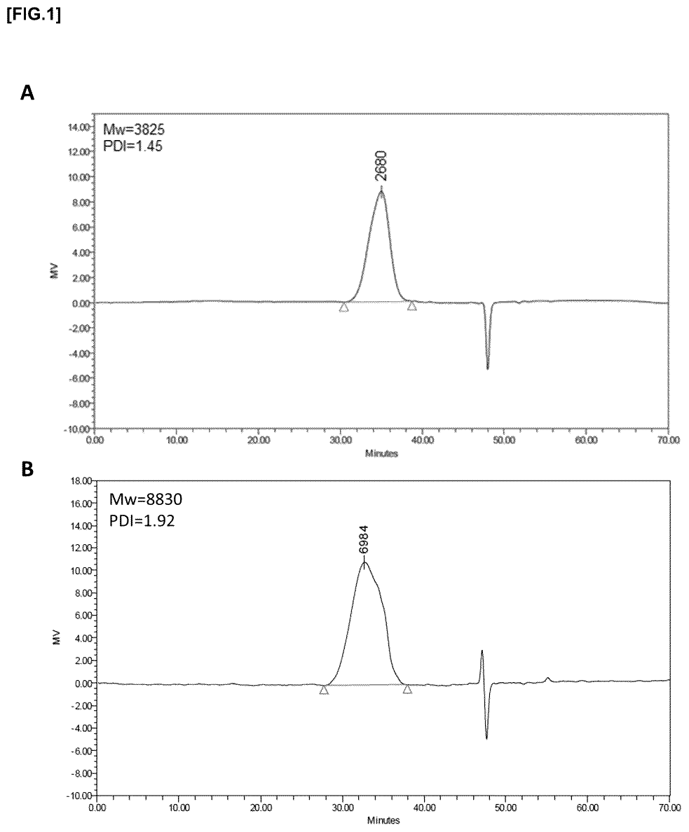

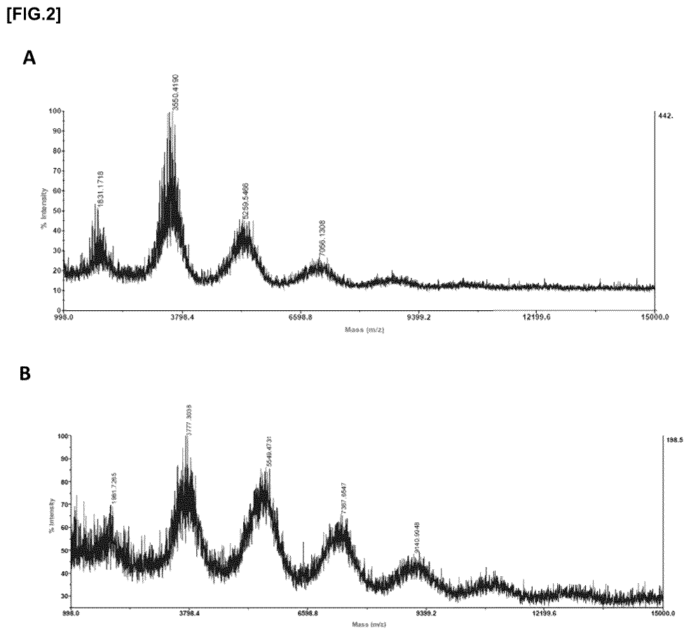

As shown in FIG. 1, a gamma-cyclodextrin oligomer (Oligo-HPGCD)having an average molecular weight of 3.8 kDa and 8.8 kDa was prepared. As shown in FIG. 2, the successful formation of oligomers of gamma-cyclodextrin could be validated through matrix-assisted laser desorption ionization time of flight mass spectrometer.

### Example 2. Effects on Prevention and Reversal of Protein Aggregation

Thioflavin T (ThT) is a fluorescent dye widely used for monitoring protein zo aggregation. To investigate the effects of gamma-cyclodextrin oligomers on preventing protein aggregation, we used amyloid beta (1-42), alpha-synuclein, and Tau (huTau441) proteins. The stock solution of ThT (1 mM) was prepared by dissolving ThT in phosphate-buffered saline (e.g., 10 mg of ThT in 31 mL of PBS) and then passing through 0.22 pm PES membrane filter. The stock solution was protected from light and stored at room temperature. To stock solutions of each protein were prepared, which were centrifuged at 20,000g for 10 minutes at 4° C. to remove any large aggregates before use. The concentrations of amyloid beta, alpha-synuclein, and Tau stock solutions were about 60 μM, 55 μM, and 40 μM, respectively. The concentration of cyclodextrin stock solution was 40 mg/mL. For prevention assays, ThT stock solution (5 μL), protein stock solution (85 μL), and cyclodextrin stock solution (5 μL) were added into a 96-well microplate and sealed. Fluorescence intensity was measured at 37° C. with Ex/Em=440 nm/485 nm every 60 minutes with 20 seconds of shaking between reads. For reversal assays, protein stock solutions incubated at 37° C. for 7 days were prepared firstly. Then, the protein stock solutions were incubated with cyclodextrins at 37° C. for 24 hours in the presence of ThT.

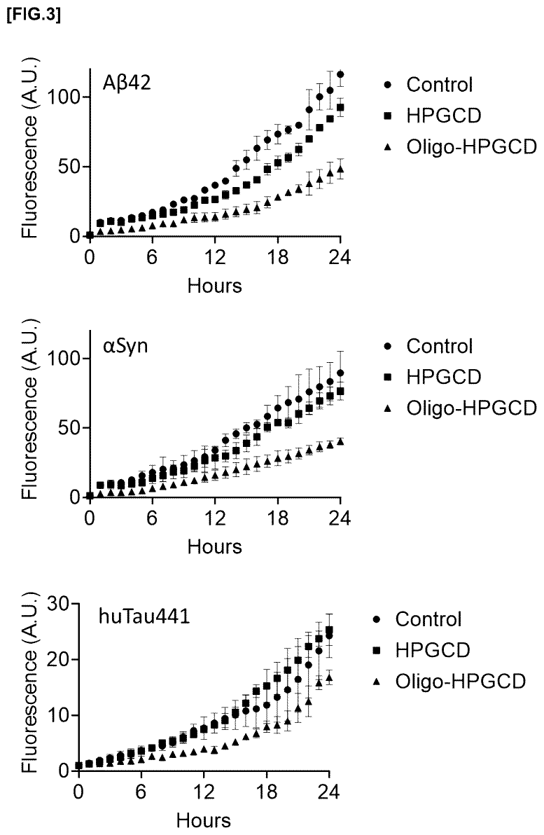

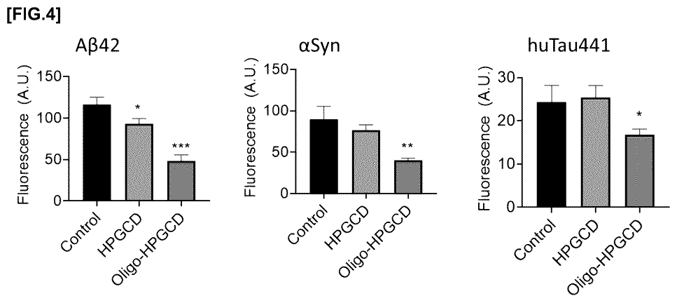

As shown in FIG. 3, cyclodextrins showed prevention of protein aggregation wherein gamma-cyclodextrin oligomer (Oligo-HPGCD group) showed more significant anti-aggregation effect compared to cyclodextrin monomer (HPGCD group). As shown in FIG. 4, Oligo-HPGCD exhibited 58.3% reduction in amyloid beta (Aβ) aggregation, 54.9% 10 reduction in alpha-synuclein (αSyn) aggregation, and 30.7% reduction in Tau (huTau441) aggregation as measured by the fluorescence of ThT.

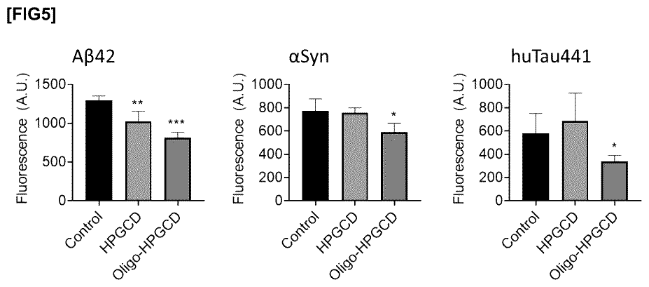

As shown in FIG. 5, Oligo-HPGCD exhibited 37.4% reversal of amyloid beta (Aβ) aggregation, 23.7% reversal of alpha-synuclein (αSyn) aggregation, and 41.7% reversal of Tau (huTau441) aggregation as measured by the fluorescence of ThT.

The results show that cyclodextrins can prevent or reverse protein aggregation and the gamma-cyclodextrin oligomer composition shows superior efficacy in preventing or reversing protein aggregation compared to cyclodextrin monomer.

### Example 3. Effects of Average Molecular Weights of Cyclodextrins

To investigate the effects of molecular weights of cyclodextrins, oligomers with zo average molecular weights of 3.8 kDa, 8.9 kDa, 13.4 kDa, and 85.2 kDa were prepared, which were comprised of about 2.5, 5.9, 8.9, and 56.8 cyclodextrin monomers, respectively. The average molecular weight of cyclodextrin monomer used here (HPGCD) is about 1500 Da. The stock solutions of each cyclodextrin were prepared at a concentration of 40 mg/mL. For prevention assays, ThT stock solution (5 μL), amyloid beta stock solution (85 μL), and cyclodextrin stock solution (5 μL) were added into a 96-well microplate and sealed. Fluorescence intensity was measured at 37° C. with Ex/Em=440 nm/485 nm at 24 hours post-incubation.

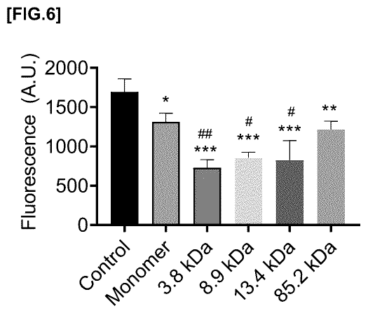

As shown in FIG. 6, gamma-cyclodextrin oligomers with average molecular weights of 3.8 kDa, 8.9 kDa, and 13.4 kDa showed the most significant anti-aggregation effects. On the other hand, gamma-cyclodextrin oligomer with an average molecular weight of 85.2 kDa showed compromised efficacy.

The results show that the gamma-cyclodextrin oligomer composition with an optimal molecular weight should be used to maximize the effects of preventing amyloid beta aggregation. As demonstrated herein, gamma-cyclodextrin oligomers with average molecular weights from about 2.5 kDa to about 50 kDa, or gamma-cyclodextrin oligomer species comprised of from 2 to 50 cyclodextrin monomers, could be optimally used for preventing protein aggregation.

### Example 4. Effects on Cytotoxicity Induced by Protein Aggregation or Deposition

To investigate the effects of cyclodextrins on cytotoxicity induced by protein aggregation or deposition, SH-SY5Y and ARPE-19 cells were exploited. Amyloid beta 1-42 (Aβ42) was dissolved in distilled water at a concentration of 1 mM, vortexed for 30 seconds, and then incubated at 37° C. for 7 days to induce pre-aggregation. SH-SY5Y cells were maintained using DMEM containing 10% fetal bovine serum, 1% Penicillin-Streptomycin, and retinoic acid (50 μM) to induce differentiation into neuron-like cells. Media was changed total three times (1, 3, and 5 days post-seeding) and the cells were seeded for experiments on the next day. ARPE-19 cells were maintained using DMEM/F12 media containing 10% fetal bovine serum, 1% Penicillin-Streptomycin, 1% HEPES, and 2.5 mM L-Glutamine. The cells were seeded in a 96-well plate at a cell zo density of 5×10⁴ cells per well. On the next day, the cells were treated with pre-aggregated Aβ42 at a concentration of 25 μM for 48 hours in the presence of cyclodextrins (1, 2.5, and 5 mg/mL). Cellular viability was measured using Cell Counting Kit-8 (CCK-8) and measuring the absorbance at 450 nm.

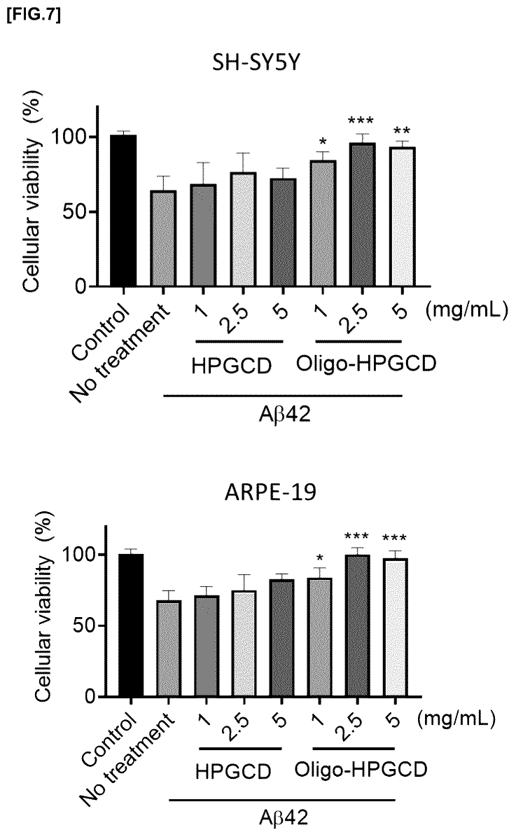

As shown in FIG. 7, the Aβ42 aggregate induced cytotoxicity in SH-SY5Y and ARPE-19 cells, inducing reduction in cell viability to 64.2% and 67.9%, respectively. We also observe that incubation with Oligo-HPGCD significantly reduced cytotoxicity induced by Aβ42, whereas HPGCD monomer did not show such effect.

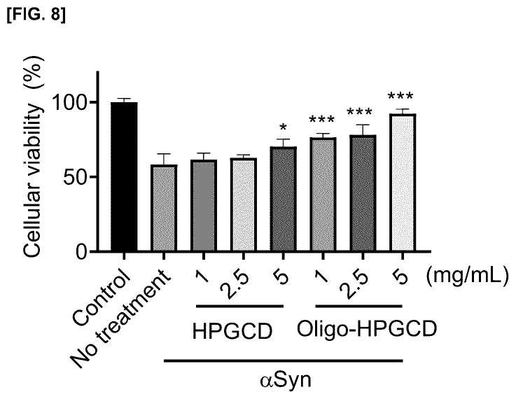

Using the SH-SY5Y cells, the cytotoxicity of aggregated alpha-synuclein (αSyn) was evaluated. The cells were treated with pre-aggregated αSyn (1 μM) for 24 hours in the presence of cyclodextrins (1, 2.5, and 5 mg/mL). As shown in FIG. 8, the αSyn aggregate induced cytotoxicity in SH-SY5Y cells, inducing reduction in cell viability to 58.3%. We observed that incubation with Oligo-HPGCD significantly reduced cytotoxicity induced by αSyn, whereas HPGCD monomer was less effective.

Then, we investigated the effects of molecular weights of gamma-cyclodextrin oligomers on the cytotoxicity induced by protein aggregates. We used Oligo-HPGCD with molecular weights of 3.8 kDa and 85.2 kDa and Oligo-GCD with molecular weights of 4.1 kDa and 99.5 kDa. The SH-SY5Y cells were treated with pre-aggregated Aβ42 for 48 hours in the presence of cyclodextrins (5 mg/mL).

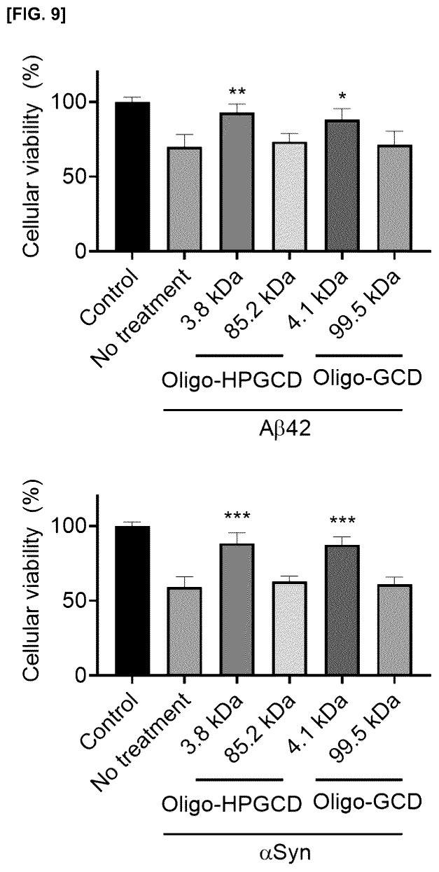

As shown in FIG. 9, the incubation with Oligo-HPGCD with molecular weights of 3.8 kDa and Oligo-GCD with molecular weights of 4.1 kDa significantly reduced cytotoxicity induced by protein aggregates. However, such effect was compromised using Oligo-HPGCD of 85.2 kDa and Oligo-GCD of 99.5 kDa, plausibly due to their large size and inability to optimally prevent protein aggregation.

The results demonstrate that the gamma-cyclodextrin oligomer with optimal molecular weights (e.g., between 2.5 kDa and 50 kDa) can prevent cytotoxicity induced by protein aggregation or deposition in various cell lines, and such effect can be compromised using gamma-cyclodextrin oligomer composition with molecular weights out of the range.

### Example 5. Effects of Cyclodextrins on Cytotoxicity and Membrane Disruption

To investigate the effects of cyclodextrins on cytotoxicity induced by cyclodextrins, we exploited hydroxypropyl-beta-cyclodextrin (HPBCD), hydroxypropyl-gamma-cyclodextrin (HPGCD), HPBCD oligomer (Oligo-HPBCD), and HPGCD oligomer (Oligo-HPGCD). HPBCD oligomer was prepared by using hydroxypropyl-beta-cyclodextrin (HPBCD) monomers with a molar substitution value between 0.4 and 0.8. The average molecular weight of Oligo-HPBCD prepared herein was 5.3 kDa. SH-SY5Y cells were maintained using DMEM containing 10% fetal bovine serum, 1% Penicillin-Streptomycin, and retinoic acid (50 μM) to induce differentiation into neuron-like cells. Media was changed total three times (1, 3, and 5 days post-seeding) and the cells were seeded for experiments on the next day. The cells were seeded in a 96-well plate at a density of 5×10⁴ cells per well. On the next day, the cells were treated with each cyclodextrin at 2.5 mg/mL, 5 mg/mL, 10 mg/mL, and 20 mg/mL for 24 hours. Cellular viability was measured using Cell Counting Kit-8 (CCK-8) and measuring the absorbance at 450 nm.

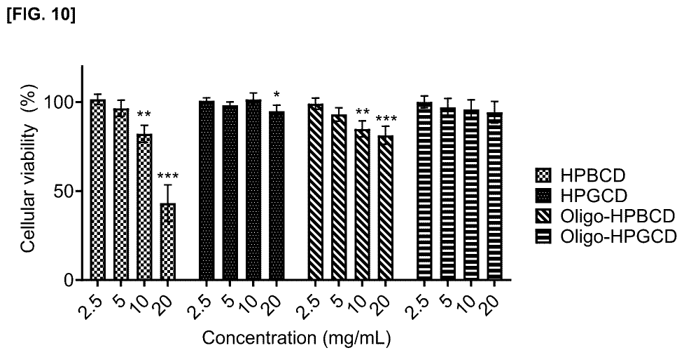

As shown in FIG. 10, the cytotoxic effect of cyclodextrins increased with increasing concentration and was high in the order of HPBCD, Oligo-HPBCD, HPGCD, and Oligo-HPGCD. HPGCD exhibited less cytotoxicity than HPBCD. Oligomer form of cyclodextrins were less cytotoxicity than their monomers. At the highest concentration (20 mg/m L), the cellular viability was 43.25%, 94.9%, 81.38%, and 94.3% for HPBCD, Oligo-HPBCD, HPGCD, and Oligo-HPGCD, respectively.

Since cyclodextrin-mediated cytotoxicity is known to be caused by excessive plasma membrane cholesterol extraction and consequential membrane disruption, we investigated cholesterol extraction from the cells. The SH-SY5Y cells were maintained seeded in a 96-well plate at a density of 5×10⁴ cells. The next day, the cells treated with cyclodextrins at a concentration of 20 mg/mL at 37° C. for 1 hour. The supernatant was collected, centrifuged at a rate of 14,000 g for 30 minutes to remove cell debris and microvesicles, and then the cholesterol concentration in the supernatant was quantified using a Cholesterol Quantification Kit (Sigma Aldrich).

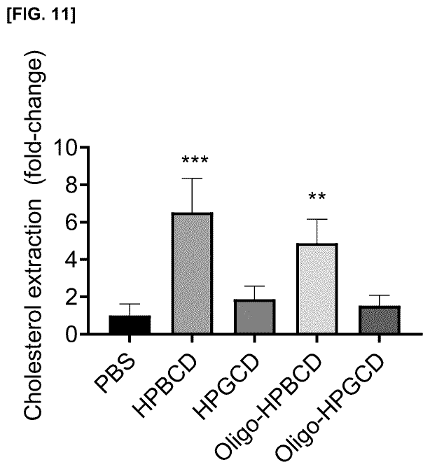

As shown in FIG. 11, the level of plasma membrane cholesterol extraction was zo high in the order of HPBCD, Oligo-HPBCD, HPGCD, and Oligo-HPGCD, wherein HPGCD and Oligo-HPGCD showed no significance compared to PBS. The level of plasma membrane cholesterol extraction correlated with cytotoxicity induced by the cyclodextrins, showing that Oligo-HPGCD do not extract plasma membrane cholesterol or disrupt the plasma membrane, thereby significantly lowering the cytotoxicity.

### Example 6. Effects on Inflammation Induced by Protein Aggregation

To investigate the effects of cyclodextrins on protein aggregate-induced inflammation, the differentiated SH-SY5Y cells were seeded in a 96-well plate at a density of 5×10⁴ cells per well. The pre-aggregated Aβ42 was treated at a concentration of 25 μM for 24 hours with or without cyclodextrins and enzyme-linked immunosorbent assay (ELISA) was performed to measure inflammatory cytokines secreted by the cells. Hydroxypropyl-gamma-cyclodextrin (HPGCD), hydroxypropyl-beta-cyclodextrin (HPBCD), HPBCD oligomer (Oligo-HPBCD, 5.3 kDa), HPGCD oligomer (Oligo-HPGCD, 3.8 kDa), and GCD oligomer (Oligo-GCD, 4.1 kDa) were treated at a concentration of 2.5 mg/mL, which did not cause cell viability loss. GCD oligomer was prepared by using gamma-cyclodextrin (GCD) monomers.

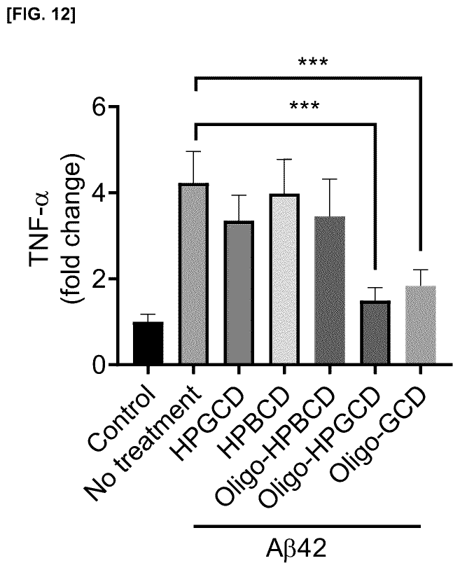

As shown in FIG. 12, the level of secreted TNF-α was significantly increased after the treatment of Aβ42, wherein Oligo-HPGCD and Oligo-GCD significantly lowered TNF-αsecretion as opposed to HPBCD and HPGCD that did not show significant effect.

The results show that the gamma-cyclodextrin oligomer (Oligo-HPGCD, Oligo-GCD) can effectively reduce inflammation induced by protein aggregation or deposition.

### Example 7. Evaluation of Therapeutic Efficacy in Zebrafish Model

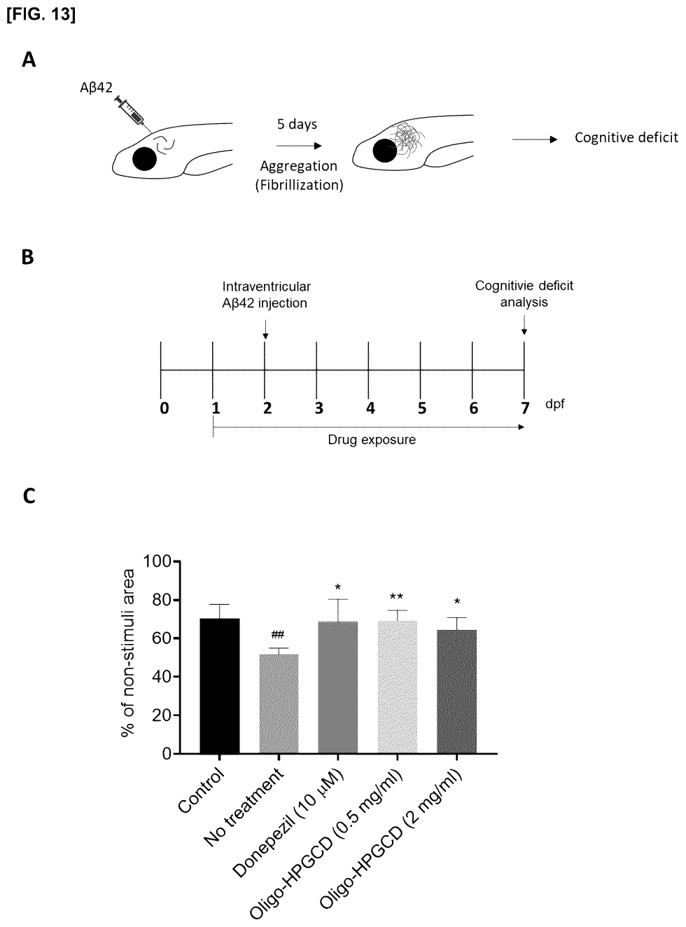

To investigate the effect of oligo-HPGCD, Alzheimer's disease was induced in Zebrafish by intraventricular injection of 5 nL of 10 μM Aβ42, which results in Aβ42 aggregation in the brain and significant cognitive deficits at 5 days post-injection of Aβ42 (FIG. 13A). The drug exposure was initiated at 1 day post-fertilization (dpf) and cognitive deficit analysis was performed at 7 dpf (FIG. 13B). Donepezil, a cholinesterase inhibitor used to improve mental function in patients with Alzheimer's disease, was used as a positive control.

As shown in FIG. 13C, the zebrafish treated with oligo-HPGCD showed significant improvement in cognitive behavior in Aβ42-injected zebrafish and the efficacy was comparable with donepezil used as positive control. The results show that gamma-cyclodextrin oligomers are effective in treating Aβ42-induced cognitive deficit in the Alzheimer's disease model of zebrafish and suggests its potential use for the treatment of proteinopathies.

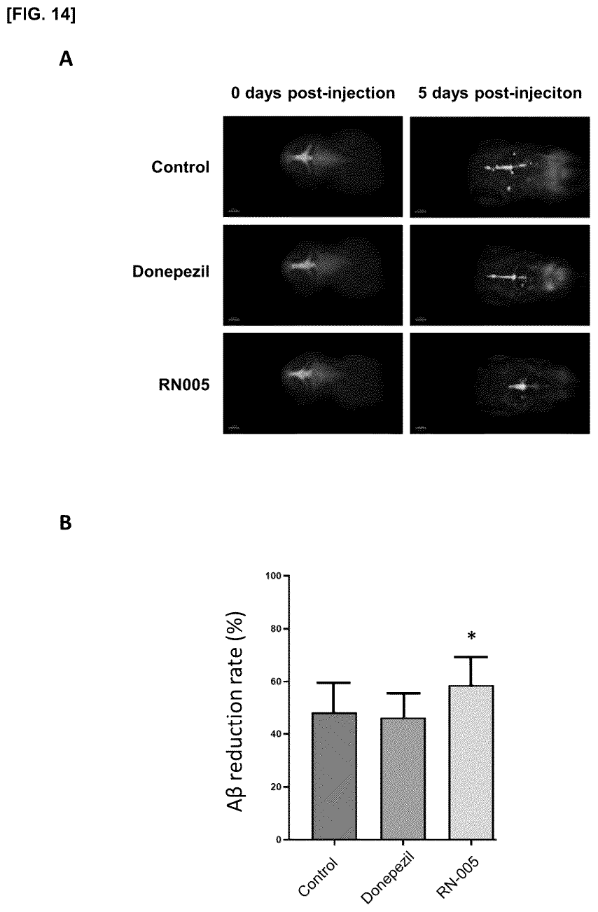

Furthermore, as shown in FIG. 14, oligo-HPGCD showed significant reduction in the amount of Aβ42 in the brain of the zebrafish at 5 days post-injection of Aβ42, whereas donepezil did not. The results show that gamma-cyclodextrin oligomers are effective in reducing Aβ42 aggregates.

1. A method of i) preventing or reducing protein aggregation, ii) reducing protein deposition, iii) reducing cytotoxicity induced by protein aggregation or deposition or deposition, or iv) alleviating or reducing inflammation induced by protein aggregation or deposition in an individual, wherein the method comprises administering a therapeutically effective amount of a pharmaceutical composition comprising a gamma-cyclodextrin oligomer to the individual, thereby preventing or reducing protein aggregation, reducing protein deposition, reducing cytotoxicity induced by protein aggregation or deposition, or alleviating or reducing inflammation induced by protein aggregation or deposition in the individual diagnosed with, suspected to have, or at risk of developing proteinopathy.

2. The method of claim 1, wherein the amount of protein aggregation in cells, tissues, or organs is reduced by at least 10% relative to the level of protein aggregation prior to treatment with the pharmaceutical composition.

3. The method of claim 1, wherein the amount of protein deposition in cells, tissues, or organs is reduced by at least 10% relative to the amount of protein deposition prior to treatment with the pharmaceutical composition.

4. The method of claim 1, wherein the level of cytotoxicity in tissues or organs induced by protein aggregation is reduced by at least 10% relative to the level of cytotoxicity prior to treatment with the pharmaceutical composition.

5. The method of claim 1, wherein the level of inflammation induced by protein aggregation or deposition in cells, tissues, or organs is reduced by at least 10% relative to the level of inflammation prior to treatment with the pharmaceutical composition.

6. The method of claim 1, wherein the average molecular weight of the gamma-cyclodextrin oligomer is between 2.5 kDa to 50 kDa.

7. The method of claim 6, wherein the gamma-cyclodextrin oligomer comprises gamma-cyclodextrin oligomer species comprised of at least 2 and at most 20 gamma-cyclodextrin monomers.

8. The method of claim 7, wherein the cyclodextrin monomer is gamma-cyclodextrin or its derivatives.

9. The method of claim 8, wherein the cyclodextrin derivative is hydroxypropyl-gamma-cyclodextrin.

10. The method of claim 9, wherein the hydroxypropyl-gamma-cyclodextrin has a molar substitution value between 0.2 and 0.9.

11. A method of treating proteinopathy in an individual diagnosed with, suspected to have, or at risk of developing proteinopathy, wherein the method comprises zo administering a therapeutically effective amount of a pharmaceutical composition comprising a gamma-cyclodextrin oligomer to the individual.

12. The method of claim 11, wherein the treating comprises i) preventing or reducing protein aggregation, ii) reducing protein deposition, iii) reducing cytotoxicity induced by protein aggregation or deposition or deposition, or iv) alleviating or reducing inflammation induced by protein aggregation or deposition in the individual.

13. The method of claim 11, wherein the proteinopathy is a taupathy.

14. The method of claim 13, wherein the taupathy is selected from a group consisting of Parkinson's disease, Alzheimer's disease, Lewy Body Dementia, Pick's disease, progressive supranuclear palsy, dementia pugilistica, parkinsonism linked to chromosome 17, Lytico-Bodig disease, tangle predominant dementia, Argyrophilic grain disease, ganglioglioma, gangliocytoma, meningioangiomatosis, subacute sclerosing panencephalitis, lead encephalopathy, tuberous sclerosis, Hallervorden-Spatz disease, lipofuscinosis, corticobasal degeneration, frontotemporal dementia, frontotemporal lobar degeneration, Huntington's disease, and age-related macular degeneration.

15. The method of claim 11, wherein the proteinopathy is synucleinopathy.

16. The method of claim 15, wherein the synucleinopathy is selected from a group consisting of Parkinson's disease, Lewy Body Dementia, multiple system atrophy, and age-related macular degeneration.

17. The method of claim 11, wherein the proteinopathy is amyloidopathy.

18. The method of claim 17, wherein the amyloidopathy is selected from a group consisting of Alzheimer's disease, and age-related macular degeneration.

19. The method of claim 11, wherein the average molecular weight of the gamma-cyclodextrin oligomer is between 2.5 kDa to 50 kDa.

20. The method of claim 19, wherein the gamma-cyclodextrin oligomer comprises cyclodextrin oligomer species comprised of at least 2 and at most 20 gamma-cyclodextrin monomers.

21. The method of claim 20, wherein the cyclodextrin monomer is gamma-cyclodextrin or its derivatives.

22. The method of claim 21, wherein the cyclodextrin derivative is hydroxypropyl-gamma-cyclodextrin.

23. The method of claim 22, wherein the hydroxypropyl-gamma-cyclodextrin has molar substitution value between 0.2 and 0.9.

24. A method of alleviating, reducing, or reversing loss of neural functions in an individual diagnosed with, suspected to have, or at risk of developing proteinopathy, wherein the method comprises administering a therapeutically effective amount of a pharmaceutical composition comprising a gamma-cyclodextrin oligomer to the individual.

25. The method of claim 24, wherein the proteinopathy comprises taupathy, zo synucleinopathy, or amyloidopathy.

26. The method of claim 24, wherein the loss of neural function comprises loss of cognitive function, autonomic function, motor function, or eye vision.

27. The method of claim 24, wherein the average molecular weight of the gamma-cyclodextrin oligomer is between 2.5 kDa to 50 kDa.

28. The method of claim 27, wherein the gamma-cyclodextrin oligomercomprises gamma-cyclodextrin oligomer species comprised of at least 2 and at most 20 gamma-cyclodextrin monomers.

29. The method of claim 28, wherein the cyclodextrin monomer is gamma-cyclodextrin or its derivatives.

30. The method of claim 29, wherein the cyclodextrin derivative is hydroxypropyl-gamma-cyclodextrin.

31. The method of claim 30, wherein the hydroxypropyl-gamma-cyclodextrin has molar substitution value between 0.2 and 0.9.
